(19) United States
(12) Patent Application Publication
(10) Pub. No.: US 2019/0078981 A1
(43) Pub. Date: Mar. 14, 2019

(54) BIO SAMPLE PRE-TREATMENT DEVICE

(71) Applicant: SK TELECOM CO., LTD., Seoul (KR)

(72) Inventors: Chin Sung PARK, Hwaseong-si (KR); Sang Ho KIM, Yongin-si (KR); Jin Seok KANG, Yongin-si (KR); Jong Sun KIM, Hwaseong-si (KR)

(21) Appl. No.: 16/084,678

(22) PCT Filed: Mar. 14, 2017
(86) PCT No.: PCT/KR2017/002760

§ 371 Date: Sep. 13, 2018

(30) Foreign Application Priority Data
Mar. 17, 2016 (KR) 10-2016-0032105

(51) Int. Cl. G01N 1/06; G01N 1/22; G01N 1/40; G01N 33/50

(52) U.S. Cl. CPC G01N 1/06; G01N 33/50; G01N 1/40; G01N 1/22

(57) ABSTRACT

A bio-sample pretreatment device according to an exemplary embodiment of the present invention includes a plurality of magnetism appliers disposed to be provided outside a prepared vessel and applying magnetism to the vessel, wherein magnetic poles of the magnetism appliers change.

### TECHNICAL FIELD

The present invention relates to a bio-sample pretreatment method and device.

### BACKGROUND ART

When a liquid sample such as blood is used, a cell lysis process may be simply started by using a centrifuge, but when a solid sample such as a stool or a tissue sample is used, a process for homogenizing or mixing the sample in a mixture with an additional reagent for pretreating the sample is additionally needed.

A most basic manual method is to pretreat the sample as follows.

1) A predetermined amount of a sample is put into a vessel.

2) A pretreatment reagent is injected.

3) The sample is sufficiently homogenized or mixed for about ten minutes by using a vortexer.

4) An impurity is precipitated by using a centrifuge.

5) A cell lysis process starts by taking a predetermined amount of an upper solution.

Techniques not using a vortexer have been developed to allow the above-noted cumbersome process to be faster and more convenient, which however just replaces the vortexer, and pre/post-processes have the same problem.

The above-described manual method may have a difficulty in manufacturing automated devices, and particularly, it has a difficulty in down-sizing the devices so that they may be applied to molecular diagnosis and point-of-care testing (POCT) devices, and subsequent additional stages have complicated problems such as non-automation.

### DISCLOSURE

### Technical Problem

The present invention has been made in an effort to provide a bio-sample pretreatment device using an electromagnet.

### Technical Solution

An exemplary embodiment of the present invention provides a bio-sample pretreatment device including a plurality of magnetism appliers disposed to be provided outside a prepared vessel and applying magnetism to the vessel, wherein magnetic poles of the magnetism appliers change.

At least one of the magnetism appliers may be oppositely arranged to the other magnetism applier with the vessel therebetween.

At least one of the magnetism appliers may be disposed with the other magnetism applier with a height difference therebetween in an vertical direction of the vessel.

Intensity of the magnetism applied by the magnetism applier may be variable.

A connector for connecting at least two of the magnetism appliers may be included.

The bio-sample pretreatment device may further include a coil for wrapping an external circumferential surface of the connector.

A direction of a current applied to the coil may be changed to change a magnetic pole of the magnetism applier.

The connector may be bent in a U shape.

A right side and a left side of the connector, wrapped by the coil, may be bent so respective ends of the connector may face each other with a vessel therebetween.

At least one tip may be formed to protrude at the magnetism applier.

The at least one tip may be provided to an upper portion of the vessel, and another tip may be provided to a lower portion of the vessel.

A height distance (d_{y}) between the tip provided to the upper portion of the vessel and the tip provided to the lower portion of the vessel may be 0.5 to 1.0 times a height of the vessel.

A thickness distance (d_{z}) between the tip provided to an upper portion of the vessel and the tip provided to a lower portion of the vessel may be 1.0 to 2.0 times a width of the vessel.

A height (h) of the tip may be 0.05 to 0.2 times a height of the vessel.

A plurality of connectors and coils may be included.

The connectors and coils may be disposed in parallel in a height direction of the vessel.

The bio-sample pretreatment device may further include a vessel disposed between the magnetism appliers.

A magnetic ball may exist inside the vessel.

A pressurizing means may be installed in an upper portion of the vessel.

A filter may be installed in a lower portion of the vessel.

The bio-sample pretreatment device may further include a temperature control means disposed to contact the vessel outside the vessel.

### Advantageous Effects

The bio-sample pretreatment device according to the exemplary embodiment of the present invention may homogenize or mix the sample by changing the external magnetism through the magnetism applier.

A very small automated device may be manufactured, and it may be applied to molecular diagnosis and POCT devices.

### DESCRIPTION OF THE DRAWINGS

FIG. 1 shows a perspective view of a bio-sample pretreatment device according to an exemplary embodiment of the present invention.

FIG. 2 shows a perspective view of a bio-sample pretreatment device in which a magnetic ball is provided on a lower portion of a vessel.

FIG. 3 shows a perspective view of a bio-sample pretreatment device in which a magnetic ball is provided on an upper portion of a vessel.

FIG. 4 shows a perspective view for showing a form in which polarities of tips change according to a direction of a current applied to a coil.

FIG. 5 shows a lateral view for showing a moving area of a magnetic ball.

FIG. 6 shows a perspective view for showing a tip.

FIG. 7 shows a front view for showing a tip.

FIG. 8 shows a perspective view bio-sample pretreatment device according to another exemplary embodiment of the present invention.

FIG. 9 shows an exploded view of a vessel.

FIG. 10 shows a perspective view of a bio-sample pretreatment device according to another exemplary embodiment of the present invention.

### MODE FOR INVENTION

Terminologies used herein are to describe only a specific exemplary embodiment, and are not to limit the present invention.

Singular forms used herein include plural forms as long as phrases do not clearly indicate an opposite meaning. A term “including” used in the present specification concretely indicates specific properties, regions, integer numbers, steps, operations, elements, and/or components, and is not used to exclude presence or addition of other specific properties, regions, integer numbers, steps, operations, elements, components, and/or a group thereof.

All terms including technical terms and scientific terms used herein have the same meaning as the meaning generally understood by those skilled in the art to which the present invention pertains unless defined otherwise.

Terms defined in a generally used dictionary are additionally interpreted as having the meaning matched to the related art document and the currently disclosed contents, and are not to be interpreted as idealized or formal meaning unless defined.

Hereinafter, embodiments of the present invention will be described in detail. However, these embodiments are merely exemplified, and the scope of protection of the present invention is not limited thereto but is defined by the appended claims.

A bio-sample pretreatment device according to an exemplary embodiment of the present invention is disposed to be provided outside a prepared vessel 10, it includes at least two magnetism appliers for applying magnetism to the vessel 10, and magnetic poles of the at least two magnetism appliers are changed.

By changing the magnetic poles of the magnetism appliers, a magnetic ball 30 provided inside the vessel 10 moves by a magnetic force generated by the external magnetism applier, it collides with a solid sample in the vessel 10 to pulverize the solid sample, and it mixes the pulverized sample with a pretreatment solution.

The magnetic ball 30 is allowed to move by changing the magnetic poles of the magnetism appliers according to an exemplary embodiment of the present invention, so a moving area of the magnetic ball 30 is wider than the case of moving a material provided inside the vessel depending on whether magnetism is applied or not, and a force applied to the magnetic ball 30 is strong to thus efficiently pulverize the solid sample and be mixed with the pretreatment solution.

At least one of the magnetism appliers may be disposed to face the other magnetism applier with the vessel 10 therebetween. According to the disposal, the magnetic ball 30 provided inside the vessel 10 may move in a right-to-left direction (i.e., x-direction) of the vessel 10.

Further, at least one of the at least two magnetism appliers may be disposed with the other magnetism applier with a height difference therebetween in a top-to-bottom direction (i.e., y-direction) of the vessel 10. According to the disposal, the magnetic ball 30 provided inside the vessel 10 may move in the top-to-bottom direction (i.e., y-direction) of the vessel 10.

In this instance, intensity of the magnetism applied by the magnetism applier may be variable

In detail, it may be a structure in which the intensity of a current is variable depending on a volume of the vessel 10, a distance between the magnetism appliers, and a size or weight of the magnetic ball 30. For example, as the volume of the vessel 10 becomes bigger, as the distance between the magnetism appliers becomes bigger, as the size of the magnetic ball 30 becomes bigger, or as the weight of the magnetic ball 30 becomes bigger, it may be a structure in which the intensity of the magnetism applied by the magnetism applier becomes variable to be bigger.

In this instance, the magnetism applied by the magnetism applier signifies a summation of intensities of the magnetism applied by the at least two magnetism appliers.

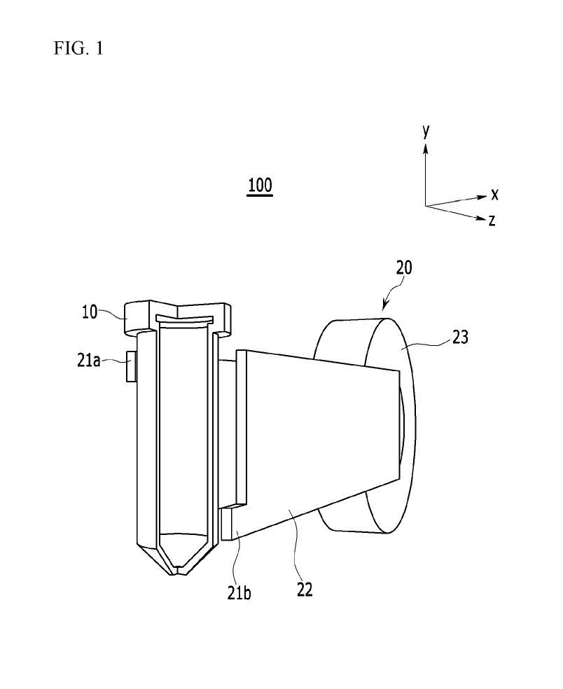

FIG. 1 shows a perspective view of a bio-sample pretreatment device according to an exemplary embodiment of the present invention. As shown in FIG. 1, a connector 22 for connecting at least two of the two or more magnetism appliers may be included. In this instance, the magnetism applier becomes an end portion of the connector 22. A coil 23 for wrapping an external circumferential surface of the connector 22 may be further included. The magnetic poles of the respective ends may be changed by changing a direction of the current applied to the coil 23. That is, the connector 22 becomes an iron core of an electromagnet 20 so that magnetism may be applied by the end portion of the connector 22.

As shown in FIG. 1, the connector 22 may have a form that is bent in a U shape. By having such the form bent in a U shape, at least one of the two or more magnetism appliers may be oppositely arranged to the other magnetism applier with the vessel 10 therebetween. A right side and a left side of a portion of the connector 22, wrapped by the coil 23, may be bent so that the respective ends of the connector 22 may face each other with the vessel 10 therebetween.

As shown in FIG. 1, at least one or more tips 21a and 21b may be respectively formed to protrude at the magnetism applier, that is, the respective ends of the connector 22. The tips 21a and 21b are formed to protrude, so magnetism is focused to strongly and efficiently move the magnetic ball 30. When the tips 21a and 21b are formed to protrude at the respective ends of the connector 22, the tips 21a and 21b become magnetism appliers.

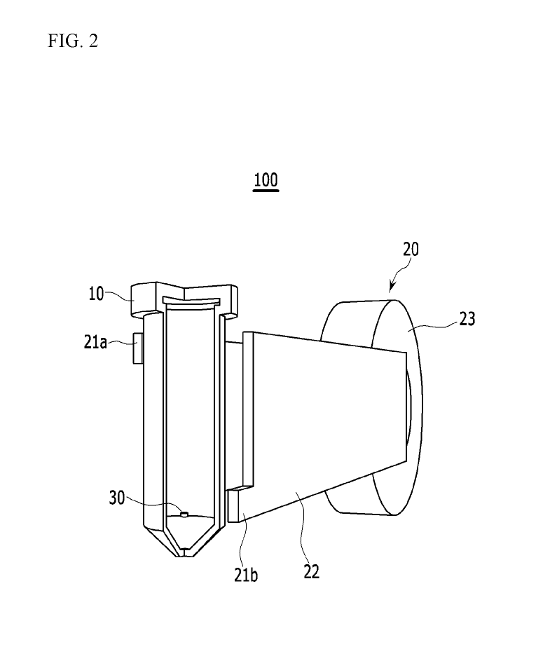

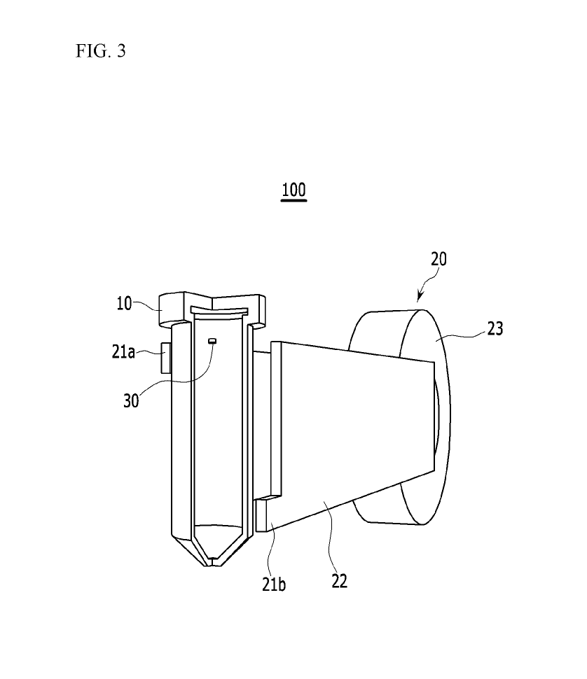

FIG. 2 shows a perspective view of a bio-sample pretreatment device 100 in which a magnetic ball 30 is provided on a lower portion of a vessel 10. FIG. 3 shows a perspective view of a bio-sample pretreatment device in which a magnetic ball 30 is provided on an upper portion of a vessel 10.

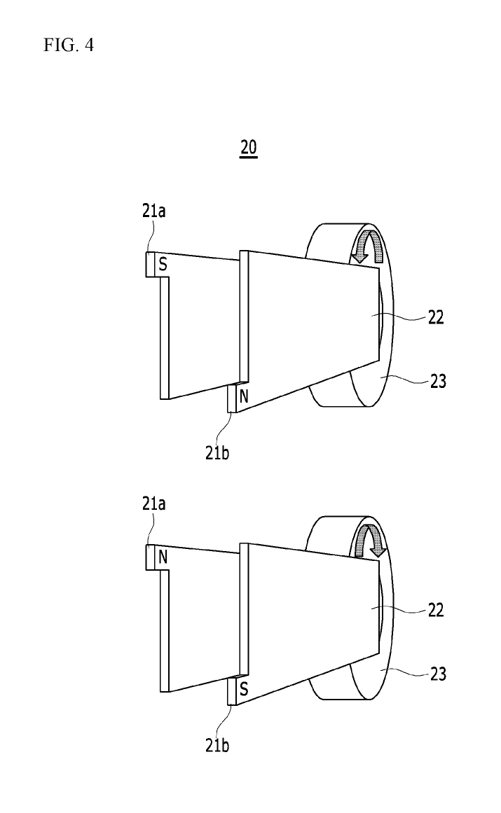

FIG. 4 shows a perspective view for showing a form in which polarities of tips 21a and 21b change according to a direction of a current applied to a coil. Regarding the upper drawing of FIG. 4, a current is applied to the coil 23 in a counterclockwise direction, and the upper tip 21a forms an S polarity and the lower tip 21b forms an N polarity. Regarding the lower drawing of FIG. 4, a current is applied to the coil 23 in a clockwise direction, and the upper tip 21a forms an N polarity and the lower tip 21b forms an S polarity.

The sizes of the tips 21a and 21b influence concentration of magnetism. To strongly and efficiently move the magnetic ball 30, it is needed to appropriately control the sizes of the tips 21a and 21b. In detail, heights (h) of the tips 21a and 21b may be 0.05 to 0.2 times the height of the vessel. When the tips 21a and 21b are very big, the magnetism may not be sufficiently concentrated. When the tips 21a and 21b are very small, there may be a problem in durability or magnetism transmission efficiency.

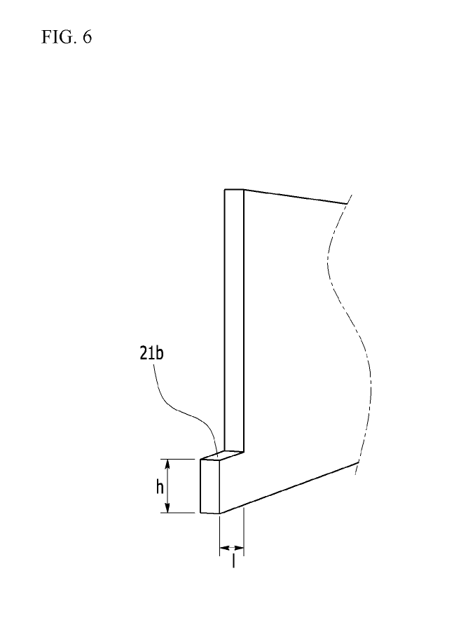

The heights (h) of the tips 21a and 21b are shown in FIG. 6 in detail. That is, the heights (h) of the tips 21a and 21b signify lengths in a height direction (i.e., y-direction).

As shown in FIG. 1, one (21a) or more of the tips may be provided to an upper portion of the vessel, and the other (21b) or more of the tips may be provided to a lower portion of the vessel. The tips 21a and 21b are provided to face each other in a diagonal direction with respect to the vessel 10, so the magnetic ball 30 provided in the vessel 10 may move in an entire area of the vessel 10.

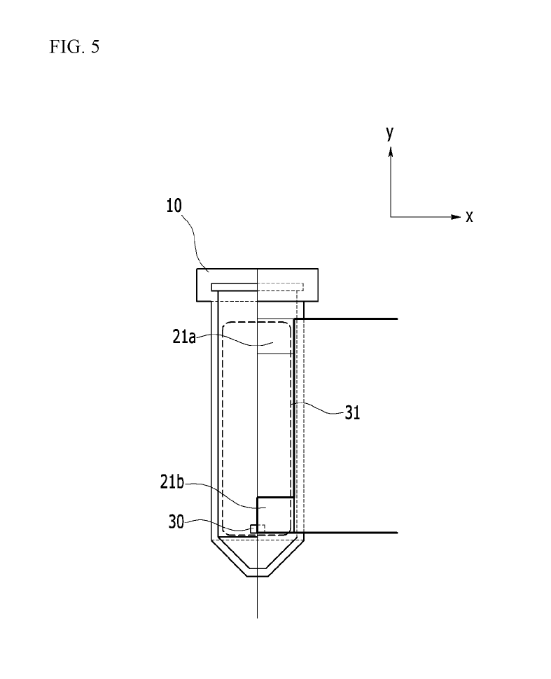

FIG. 5 shows a lateral view for showing a moving area of a magnetic ball 30. As shown in FIG. 5, by appropriately setting the positions of the tips 21a and 21b, movement is possible in the entire area of the vessel 10. In detail, a height distance (d_{y}) between the tip 21a provided to the upper portion of the vessel 10 and the tip 21b provided to the lower portion of the vessel 10 may be 0.5 to 1.0 times the vessel height. When the height distance (d_{y}) is too small or big, the magnetic ball 30 moves in a predetermined area of the vessel 10, so the sample may not be fluently pulverized or mixed.

Further, a thickness distance (d_{z}) between the tip provided to the upper portion of the vessel and the tip provided to the lower portion of the vessel may be 1.0 to 2.0 times the vessel width. When the thickness distance (d_{z}) is too big, magnetism may not be fluently transmitted to the magnetic ball 30.

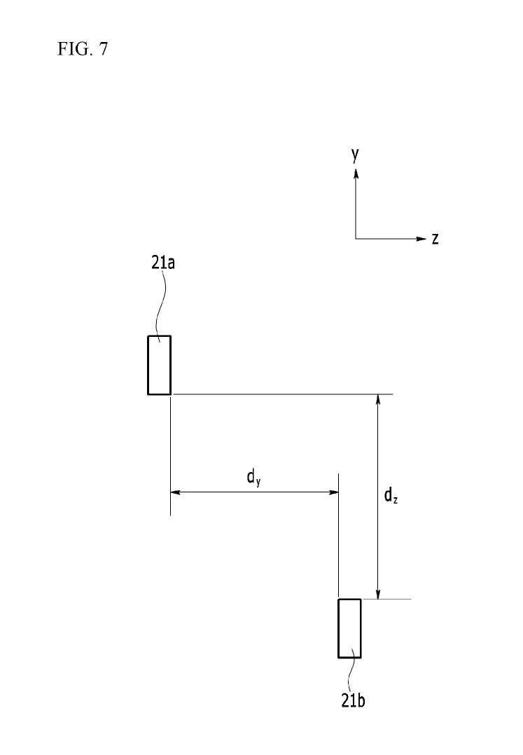

The magnetic ball 30 is allowed to move in the entire area of the vessel 10 by appropriately controlling the height distance (d_{y}) and the thickness distance (d_{z}) of the tips 21a and 21b. The height distance (d_{y}) and the thickness distance (d_{z}) are shown in detail in FIG. 7. That is, the height distance (d_{y}) signifies a gap between the tips in the height direction (i.e., y-direction), and the thickness distance (d_{z}) signifies a gap between the tips 21a and 21b in the thickness direction (i.e., z-direction).

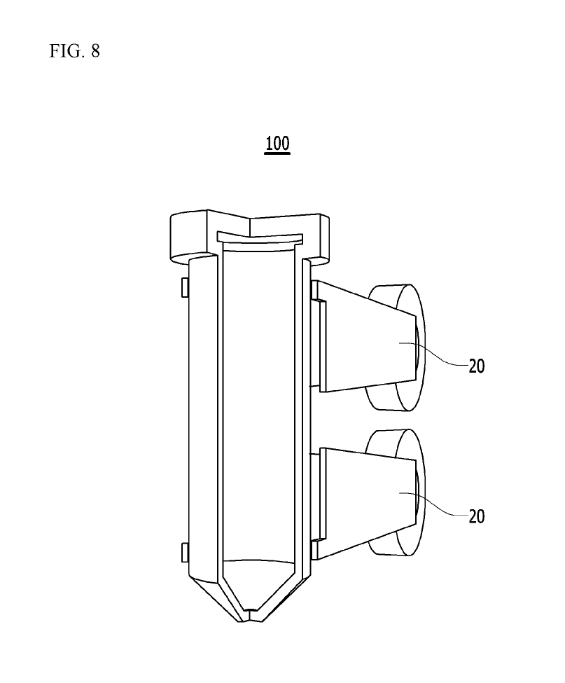

According to another exemplary embodiment of the present invention, the bio-sample pretreatment device may include a plurality of electromagnets 20 including a connector 22 and a coil 23. An example thereof is shown in FIG. 8. As shown in FIG. 8, when there are a plurality of electromagnets 20 including a connector 22 and a coil 23, they may be disposed in parallel in the height direction (i.e., y-direction) of the vessel 10. In this instance, the magnetic poles of the adjacent tips are formed to be opposite, and the intensity of magnetism applied to the respective tips is controlled, so the magnetic ball 30 may move in the entire area of the vessel 10.

Referring to FIG. 1, the bio-sample pretreatment device according to an exemplary embodiment of the present invention may further include the vessel 10 disposed between the magnetism appliers. The solid sample may be pretreated by inputting a solid sample to be treated and a pretreatment solution into the vessel 10. A cover that may be opened or closed may be formed on the upper portion of the vessel 10. The cover prevents the sample from splashing out of the vessel in the pretreatment process. However, when the vessel 10 is heated by a temperature control means 40 to be described, fine pores may be formed in the cover in order to prevent the cover from being opened by a pressure increase inside the vessel 10.

A magnetic ball 30 may be provided in the vessel 10. By changing the magnetic pole of the magnetism applier disposed outside the vessel, the magnetic ball 30 moves in the vessel 10 so that the sample may be pulverized and may be uniformly mixed with the pretreatment solution. The magnetic ball 30 may be used without limit as long as it is a magnetic ball that is affected by the N polarity and the S polarity. The shape of the magnetic ball 30 is not limited. However, to easily pulverize the solid sample, a bar-type or cylinder-type magnetic ball 30 for densely configuring the magnetic ball 30 may be used.

A pressurizing means may be mounted to the upper portion of the vessel 10. Further, a filter 50 may be mounted to the lower portion of the vessel 10.

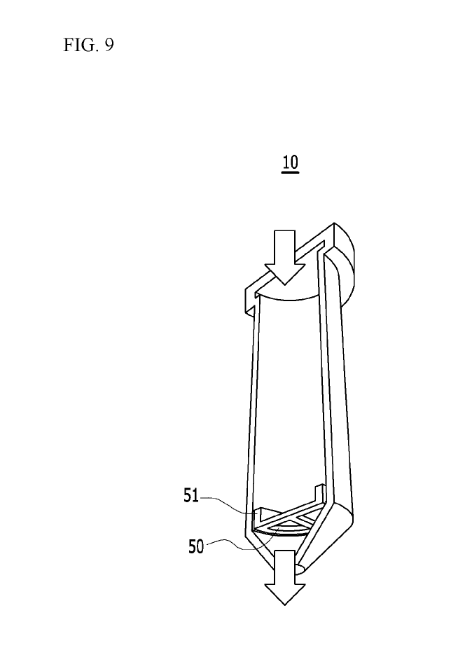

FIG. 9 shows a vessel 10 in which a filter 50 is mounted. The pretreatment solution may be pressurized by a pressurization means installed in the upper portion of the vessel 10 to allow the same to pass through the filter 50. FIG. 9 shows pressurizing the pretreatment solution in an arrow direction and allowing the same to pass through the filter 50. The filter 50 is installed in the lower portion of the vessel 10, and is fixed by a holder 51.

A pore diameter of the filter 50 may be 1 μm to 30 μm. The above-noted pores sift out an impurity in the pretreatment solution, and pathogenic viruses for extracting nucleic acid may fluently pass through the pores with this diameter.

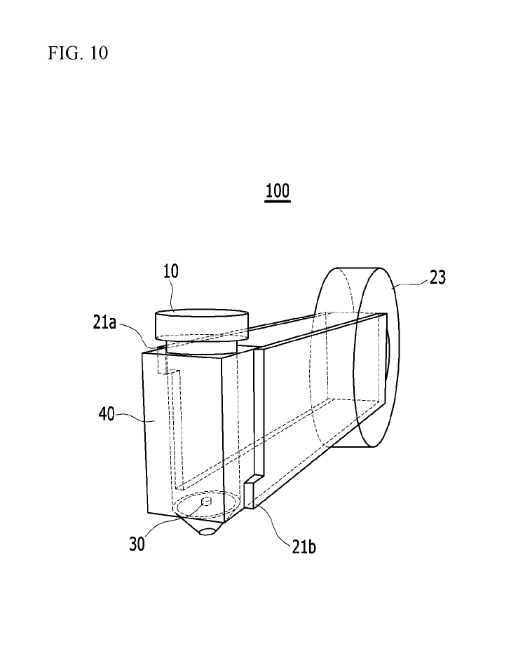

According to another exemplary embodiment of the present invention, the bio-sample pretreatment device 100 may further include a temperature control means 40 disposed to contact the vessel 10 outside the vessel 10. An example of the bio-sample pretreatment device 100 further including a temperature control means 40 is shown in FIG. 10. As shown in FIG. 10, the temperature control means 40 is disposed to contact the vessel 10, and a magnetism applier is disposed outside the temperature control means 40. The temperature control means 40 may increase or reduce the temperature inside the vessel 10 as needed. To fluently pulverize the solid sample, the temperature control means 40 may increase the internal temperature of the vessel 10, and in this instance, the temperature control means 40 may be a heater.

A method for pretreating a bio-sample by using a bio-sample pretreatment device 100 according to an exemplary embodiment of the present invention will now be described. However, the bio-sample pretreatment method just exemplifies the present invention, and the present invention is not limited thereto. Therefore, the bio-sample pretreatment method may be modified in various ways.

The bio-sample pretreatment method includes: preparing a vessel 10 into which a solid sample, a pretreatment solution, and a magnetic ball 30 are input (S10); pulverizing the solid sample and uniformly dispersing the same with a pretreatment solution by alternately changing a polarity of a magnetism applier installed outside the vessel 10 and moving the magnetic ball 30; and removing an impurity of the pretreatment solution by allowing the pretreatment solution to which the solid sample is dispersed to pass through the filter 50 installed at the lower portion of the vessel 10. In addition, the bio-sample pretreatment method may further include other stages if needed.

First, a vessel to which a solid sample, a pretreatment solution, and a magnetic ball are input is prepared (S10).

Samples for diagnosing infectious diseases by use of a molecular diagnosis method are used for the solid sample. The sample needs a pretreatment process for keeping pathogenic viruses and removing the rest of the sample. When the sample is a liquid such as urine or saliva, the pretreatment process for removing an impurity through a centrifugation process may be simply performed, and when the sample is a solid, a homogenizing or mixing process for uniformly melting the sample in the pretreatment solution is additionally required. An exemplary embodiment of the present invention aims at automatically and simply processing the homogenizing or mixing process. Representative solid samples may include a tissue, a stool, and sputum.

The pretreatment solution may use a general pretreatment solution used in the molecule diagnosis method. When the pretreatment solution and the solid sample are input, the solid sample at 0.01 to 0.1 parts by weight may be input with respect to the pretreatment solution of 100 parts by weight. When a very small amount of the solid sample is input, a yield is very low, and the pretreatment process must be repeated multiple times, which may be inefficient. When a very large amount of the solid sample is input, the homogenization or mixing may not be fluently performed. Therefore, the amount of the pretreatment solution and the solid sample may be controlled within the above-noted range.

The magnetic ball 30 moves by a magnetic force generated by an external magnetism applier to collide with the solid sample in the vessel 10 and pulverize the solid sample. The size of the magnetic ball 30 may be 0.1 mm to 5 mm. When the above-noted range is not satisfied, the solid sample may not be appropriately pulverized.

An order for inputting the solid sample, the pretreatment solution, and the magnetic ball 30 is not specifically limited. For example, the solid sample, the pretreatment solution, and the magnetic ball 30 may be input in different orders, and at least two thereof may be simultaneously input.

Next, in the stage S20, the solid sample is pulverized and is uniformly dispersed in the pretreatment solution by moving the magnetic ball 30 by alternately changing the polarity of the magnetism applier installed outside the vessel 10.

As shown in FIG. 1, the tips 21a and 22b of the electromagnet 20 are disposed with the vessel 10 therebetween. By the disposal, the magnetic ball 30 may vibrate to the right and left of the vessel 10.

Further, as shown in FIG. 1, one (21a) of the tips of the electromagnet 20 is provided to the upper portion with respect to a length direction of the vessel 10, and the other (21b) of the tips of the electromagnet 20 may be provided to the lower portion of the vessel 10. By the disposal, the magnetic ball 30 may vibrate up and down in the vessel 10. FIG. 2 shows that the magnetic ball 30 is provided to the lower portion of the vessel 10 and is adjacent to the tip 21b of the electromagnet 20. Further, FIG. 3 shows the state in which the polarities of the tips 21a and 21b are changed with each other, and the magnetic ball 30 is provided to the upper portion of the vessel 10 and is adjacent to the other 21a of the tips of the electromagnet 20. As described, the magnetic ball 30 freely moves up and down and right and left of the vessel 10 through an external electromagnet 20 to pulverize the solid sample.

The magnetic force becomes stronger as it goes to the end terminal of the electromagnet 20, so as shown in FIG. 5, the end terminals of the tips 21a and 21b may be disposed at a center with respect to the thickness direction (i.e., x direction) of the vessel 10.

By appropriately disposing the electromagnet 20 outside the vessel 10, the magnetic ball 30 is moved in the vessel 10 to thus pulverize the solid sample. FIG. 5 shows a moving area 31 in which the magnetic ball 30 moves. This is an example, and the solid sample may be pulverized by moving the magnetic ball 30 through appropriate disposal of the electromagnet 20.

The current applied to the electromagnet 20 may be 0.01 to 10 A, and the direction of the current may be changed with a period of 10 to 100 Hz. The solid sample may be efficiently pulverized by satisfying the above-noted ranges.

The pulverized solid sample is uniformly dispersed to the pretreatment solution by an agitation effect caused by the movement of the magnetic ball 30 together with the pulverization of the solid sample.

A temperature at the vessel 10 may be maintained at 30° C. to 95° C. by using means such as a temperature control means 40 installed outside the vessel 10 in the stage S20. By maintaining the temperature, the solid sample may be prevented from being transformed, and pretreatment efficiency may be increased.

Next, in the stage S30, the impurity provided in the pretreatment solution is removed by allowing the pretreatment solution into which the solid sample is dispersed to pass through the filter 50 installed on the lower side of the vessel 10.

In this instance, the pretreatment solution may be pressurized by the pressurizing means installed on an upper side of the vessel 10 to allow the same to pass through the filter. FIG. 9 shows pressurizing of a pretreatment solution in an arrow direction to allow the same to pass through a filter 50. The filter 50 is installed on the lower side of the vessel 10, and is fixed by a holder 51.

A pore diameter of the filter 50 may be 1 μm to 30 μm. The pores with the above diameter may sift the impurity in the pretreatment solution, and may allow the pathogenic viruses for extracting nucleic acids to fluently pass therethrough.

The bio-sample pretreated according to an exemplary embodiment of the present invention may be analyzed through extraction of nucleic acids. A method for extracting and analyzing nucleic acids may be a general method known to a skilled person in the art, and detailed descriptions thereof will be omitted in the present specification.

The following examples illustrate the present invention in more detail. However, an exemplary embodiment to be described below is just an exemplary embodiment of the present invention, and the present invention is not limited thereto.

### Exemplary Embodiment—Pretreatment Using Electromagnet

A 200 mg tube-type stool sample and 1 ml of a pretreatment solution are input to the vessel together with a magnetic ball (diameter: 2 mm), an electromagnet is installed, pretreatment is performed with a current of 3 A, an oscillation frequency of 25 Hz, and a temperature of 85° C. for five minutes, and 200 μl of a solution is obtained through a membrane filter (pore diameter: 2 μm).

### COMPARATIVE EXAMPLE

### Pretreatment Using a Vortexer

A 200 mg tube-type stool sample and 1 ml of a pretreatment solution are input to the vessel, and they are mixed until the sample is completely homogenized or mixed by using a vortexer. The homogenized or mixed sample is heated for five minutes at a temperature of 95° C. by using a heat block, and it is centrifuged for one minute at 14,500 rpm by using a centrifuge to thereby obtain 200 μl of a solution.

### EXPERIMENTAL EXAMPLE

### Nucleic Acid Extraction and Analysis

The respective 200 μl solutions pretreated according to the exemplary embodiment and the comparative example and a 10 μl magnetic bead solution are provided into a 500 μl lysis/binding buffer, and are reacted for five minutes. Magnetic particles are moved into a 750 μl first washing buffer and are mixed for one minute by using a magnetic rod. After they are mixed, the magnetic particles are moved into a second 750 μl washing buffer and are mixed for one minute by using a magnetic rod. After they are mixed, the magnetic particles are separated by using a magnetic rod, and they are dried in the air for one minute. The dried magnetic particles are input to a 200 μl elution buffer and mixed for one minute. After they are mixed, the magnetic particles are removed by using a magnetic rod, and the rest of the solution is used for nucleic acid analysis. To minimize a deviation between comparison methods according to the nucleic acid extracting method, an NP968 device (TianLong, China) that is a device for automatically performing the nucleic acid extracting process is used, and results are expressed in Table 1.
Nucleic acid analysis method: Yields and purities of the nucleic acid extracted by using a real-time PCR, a spectrophotometer, and a fluorometer (Real-time PCR: AdvanSure CD real-time PCR (LG LifeSciences, Korea), Spectrophotometer: Nanodrop 2000C (Thermo Scientific, Uganda), and Fluorometer: Qubit 2.0, Qubit dsDNA HS Assay Kit (Invitrogen, Uganda)).

TABLE 1

| Pre- | PCR (Ct) | | Spectrophotometer | | | Fluorometer |
|---|---|---|---|---|---|---|
| treatment | Toxin | Toxin | Yield | Purity | Purity | Yield |
| method | A | B | (ng/μl) | 260/280 | 260/230 | (ng/ml) |
| Exemplary | 22.57 | 23.18 | 101.5 | 1.53 | 0.6 | 9900 |
| embodiment | | | | | | |
| Comparative | 21.55 | 21.65 | 24.5 | 2.06 | 1.45 | 1740 |
| example | | | | | | |

As expressed in Table 1, it is found that the homogenization or mixing of the sample using an electromagnet and the pretreatment performance are excellent compared to the comparative example using a vortexer.

The present invention is not limited to the exemplary embodiments, but may be implemented in various different forms. It may be understood by those skilled in the art to which the present invention pertains that the present invention may be implemented in other specific forms without changing the spirit or essential features thereof. Therefore, it should be understood that the above-mentioned embodiments are not restrictive but are exemplary in all aspects.

1. A bio-sample pretreatment device comprising
a plurality of magnetism appliers disposed to be provided outside a prepared vessel and applying magnetism to the vessel,
wherein magnetic poles of the magnetism appliers change.

2. The bio-sample pretreatment device of claim 1, wherein
at least one of the magnetism appliers is oppositely arranged to the other magnetism applier with the vessel therebetween.

3. The bio-sample pretreatment device of claim 1, wherein
at least one of the magnetism appliers is disposed with the other magnetism applier with a height difference therebetween in an vertical direction of the vessel.

4. The bio-sample pretreatment device of claim 1, wherein
intensity of the magnetism applied by the magnetism applier is variable.

5. The bio-sample pretreatment device of claim 1, wherein
a connector for connecting at least two of the magnetism appliers is included.

6. The bio-sample pretreatment device of claim 5, further comprising
a coil for wrapping an external circumferential surface of the connector.

7. The bio-sample pretreatment device of claim 6, wherein
a direction of a current applied to the coil is changed to change a magnetic pole of the magnetism applier.

8. The bio-sample pretreatment device of claim 5, wherein
the connector is bent in a U shape.

9. The bio-sample pretreatment device of claim 8, wherein
a right side and a left side of the connector, wrapped by the coil, are bent so respective ends of the connector face each other with a vessel therebetween.

10. The bio-sample pretreatment device of claim 5, wherein
at least one tip is formed to protrude at the magnetism applier.

11. The bio-sample pretreatment device of claim 10, wherein
the at least one tip is provided to an upper portion of the vessel, and another tip is provided to a lower portion of the vessel.

12. The bio-sample pretreatment device of claim 11, wherein
a height distance (d_{y}) between the tip provided to the upper portion of the vessel and the tip provided to the lower portion of the vessel is 0.5 to 1.0 times a height of the vessel.

13. The bio-sample pretreatment device of claim 11, wherein
a thickness distance (d_{z}) between the tip provided to an upper portion of the vessel and the tip provided to a lower portion of the vessel is 1.0 to 2.0 times a width of the vessel.

14. The bio-sample pretreatment device of claim 11, wherein
a height (h) of the tip is 0.05 to 0.2 times a height of the vessel.

15. The bio-sample pretreatment device of claim 6, wherein
a plurality of connectors and coils are included.

16. The bio-sample pretreatment device of claim 15, wherein
the connectors and coils are disposed in parallel in a height direction of the vessel.

17. The bio-sample pretreatment device of claim 1, further comprising
a vessel disposed between the magnetism appliers.

18. The bio-sample pretreatment device of claim 17, wherein
a magnetic ball exists inside the vessel.

19. The bio-sample pretreatment device of claim 17, wherein
a pressurizing means is installed in an upper portion of the vessel.

20. The bio-sample pretreatment device of claim 17, wherein
a filter is installed in a lower portion of the vessel.

21. The bio-sample pretreatment device of claim 17, further comprising
a temperature control means disposed to contact the vessel outside the vessel.
